(19) United States
(12) Patent Application Publication
(10) Pub. No.: US 2011/0105912 A1
(43) Pub. Date: May 5, 2011

(54) CEREBRAL AUTOREGULATION INDICES

(76) Inventors: Ronald A. Widman, Macomb, MI (US); Oleg Gonopolskiy, West Bloomfield, MI (US); Arik Anderson, Birmingham, MI (US); Bruce J. Barrett, Birmingham, MI (US)

(21) Appl. No.: 12/940,633

(22) Filed: Nov. 5, 2010

Related U.S. Application Data
(60) Provisional application No. 61/258,470, filed on Nov. 5, 2009.

(51) Int. Cl. A61B 5/0205

(52) U.S. Cl. 600/483

(57) ABSTRACT

Monitoring cerebral autoregulation may include determining one or more autoregulation indices incorporating cerebral blood flow and blood pressure measurements and/or indices. Measurement techniques may be invasive or non-invasive. Various combinations of data, e.g., oximeter data, electrocardiogram data, blood pressure data, hemoglobin data, and heart rate data, may be used to create various indices. Many of the indices may be based on correlations of data. A display may indicate several of the indices.

### CROSS-REFERENCE TO RELATED APPLICATIONS

This application claims priority to U.S. Provisional Application No. 61/258,470, filed on Nov. 5, 2009, which is incorporated herein by reference in its entirety.

### BACKGROUND

Cerebral autoregulation is the mechanism in humans that ensures a consistent cerebral blood flow (CBF) over a range of cerebral perfusion pressure (CPP). In a healthy subject, the cerebral arteries and arterioles constrict or dilate to maintain CBF during changes in arterial blood pressure, thereby ensuring adequate blood flow and protecting against excessive blood flow which can result in brain swelling or edema. Monitoring of CBF in the face of changing CPP can delineate the optimal range of blood pressure where autoregulation is maintained. A number of disease states, including traumatic brain injury, stroke, meningitis, cardiac arrest and other brain insults can impair cerebral autoregulation by limiting or shifting the optimal range of CPP where CBF is relatively constant. Various therapies and interventions can also impair cerebral autoregulation, such as cardiopulmonary bypass and hypothermia. Continuous monitoring of the autoregulatory state is needed to protect against brain hypoxia due to hypoperfusion and cerebral edema due to over perfusion.

### BRIEF DESCRIPTION OF THE DRAWINGS

FIG. 1 is a schematic illustration of a system for diagnosing cerebrovascular autoregulation.

FIG. 2 is from the article D. Rassi, A Mishin, Time Domain Correlation Analysis of Heart Rate Variability in Preterm Infants, Early Human Development (2005) 81, 341, and illustrates the anti-correlation between blood pressure and heart rate in preterm infants.

FIG. 3 illustrates the relationship between mean arterial pressure (MAP) and cerebral blood flow (CBF) at low and high CMRO2.

FIG. 4 illustrates the relationship between mean arterial pressure (MAP) and CBF/CMRO2 at low and high CMRO2.

FIG. 5 illustrates the relationship between rSO2 and MAP.

FIG. 6 illustrates an embodiment for displaying cerebral oximetry autoregulation indices (COx) at various blood pressures.

### DETAILED DESCRIPTION

The contents of all references, including articles, published patent applications and patents referred to anywhere in this specification are hereby incorporated by reference. Described below are various systems and methods of monitoring autoregulation in a patient.

Cerebral Autoregulation Generally

Cerebral autoregulation is a body's internal mechanism for regulating the balance of cerebral blood flow and cerebral perfusion pressure. The direct measurement of cerebral blood flow and cerebral perfusion pressure require invasive techniques. Further, invasive and/or intermittent techniques may interfere with the autoregulation process that is being measured. Therefore, non-invasive and continuous methods are sought to create an index or indices of autoregulation that are an accurate representation of the actual autoregulation.

Autoregulatory indices describe the relationship between cerebral blood flow and cerebral perfusion pressure. An autoregulatory index may include any combination of direct measurements and representative indices for components of flow or pressure.

### Definitions

Cerebral Blood Flow (CBF): An element in a calculation of an autoregulation index may be data reflecting cerebral blood flow (CBF). An exemplary process for calculating an autoregulation index may include the use of a direct measurement of CBF, or may use a CBF index representing changes in CBF. One summary of measurement methods using CBF or a CBF index can be found in the article Panerai R B, Assessment of Cerebral Pressure Autoregulation in Humans—A Review of Measurement Methods, Physiol Meas 1998; 19(3):305-338.

Near-Infrared Spectroscopy (NIRS): Near infrared spectroscopy is a non-invasive method of measurement of hemoglobin using signals in the near infrared spectrum. Near-infrared signals are directed at a region of brain tissue and a detector measures the intensity of transmitted or reflected signals at different wavelengths.

Oximetry: Oximetry is a non-invasive method of measuring oxygenation of hemoglobin, wherein light is passed through a portion of a body and the absorbance of the light is measured. Light of multiple wavelengths may be used to indicate a difference in absorption between the light at the various wavelengths corresponding with oxygenation.

Arterial Blood Pressure (ABP): Arterial blood pressure is the pressure of blood in the arteries, generally measured non-invasively.

Mean Arterial Pressure (MAP): The Mean Arterial Pressure is the average arterial pressure during a cardiac cycle. MAP may be used as an index or in the calculation of an index for cerebral perfusion pressure.

Cerebral Perfusion Pressure (CPP): Cerebral Perfusion Pressure is the average pressure of blood in the brain. It can be calculated by subtracting either the intracranial pressure or the pressure of brain venous outflow from MAP.

Diffuse Optical Tomography: Diffuse optical tomography uses continuous amplitude-modulated near-infrared light of one wavelength (chosen to be at or near the isobestic point of hemoglobin) injected through the skin, with light in the wavelength collected at multiple positions and multiple distances from the source, to determine absorption and scattering of the light. These measurements can be compared over time to estimate red blood cell movement as an index of CBF.

Pulse contour analysis: A cerebral oximetry sensor may be used to measure characteristics of the pulsatile component of total hemoglobin (or cerebral blood volume) as an index of cerebral blood flow resulting from arterial oscillations. (See, e.g., Themelis G et al., Near-Infrared Spectroscopy Measurement of The Pulsatile Component of Cerebral Blood Flow and Volume From Arterial Oscillation, J Biomed Optics 2007; 12(1): 014033.) In this implementation, each heart beat causes a change in blood pressure which can be measured peripherally using a fluid-filled catheter and pressure transducer to convert pressure changes to electrical changes. The slope of the change in pressure resulting from each heart beat represents the change in blood flow per time unit. Thus, the time derivative of blood pressure (or total hemoglobin) is proportional to blood flow and can be used to derive changes in flow. (See, e.g., Remington J W et al., Volume Elasticity Characteristics of The Human Aorta and The Prediction of Stroke Volume from the Pressure Pulse, Am J Physiol 1948; 153: 198-308). Several products on the market today rely on the measurement of pressure change and area to derive cardiac output (flow) from the arterial pressure waveform, for example, products like LiDCO Plus (LiDCO Ltd.), or FloTrac System (Edwards Lifesciences).

System for Diagnosing Cerebrovascular Autoregulation

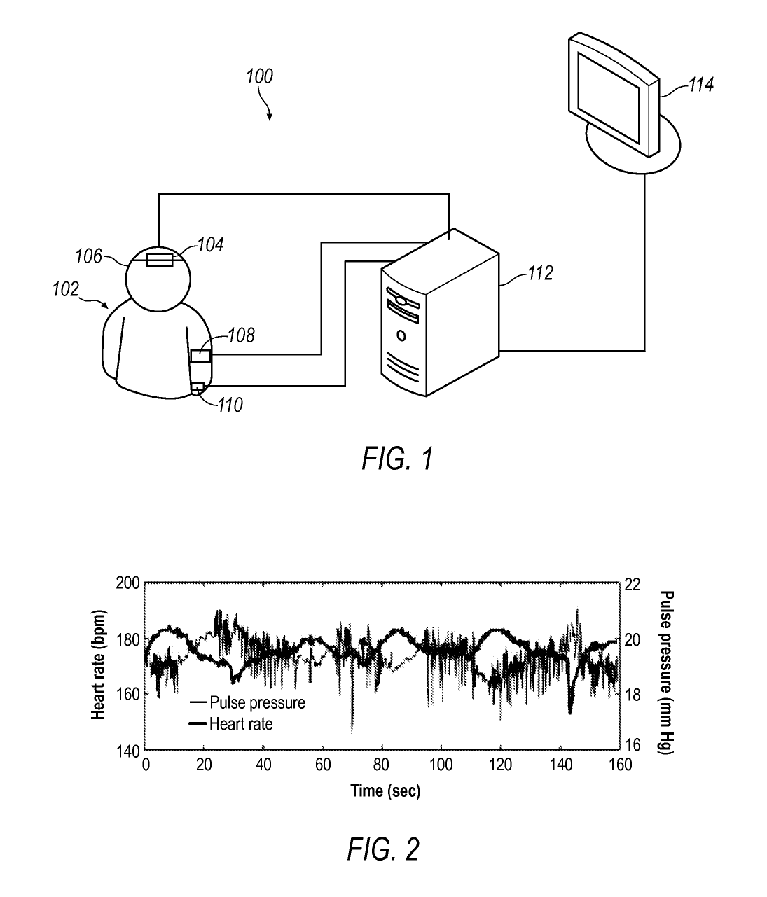

FIG. 1 illustrates one example of a system 100 for diagnosing cerebrovascular autoregulation of a patient 102. System 100 includes a sensor 104 that is arranged proximate to an external position of the patient's head 106. In one example, sensor 104 is a cerebral oximeter. A blood pressure monitoring device 108 is attached to the patient, for example, to a patent's arm. Further, a pulse oximeter sensor 110 may be attached to a patent's hand or finger, as discussed in greater detail below. A signal processing unit 112 is in communication with cerebral oximeter 104 and with blood pressure monitoring device 108. In one example, the cerebral oximeter obtains oxygen content measurements of blood within the patient's brain. Signals from cerebral oximeter 104 may be processed internally within cerebral oximeter 104 and/or processed by signal processing unit 112. For example, the oxygen content measurements of blood within the patient's brain is taken at a plurality of times by cerebral oximeter 104 to input an oxygen content signal to signal processing unit 112.

Blood pressure monitoring device 108 obtains arterial blood pressure measurements of patient 102 at a plurality of times substantially synchronously with the oxygen content measurements and outputs an arterial blood pressure signal to signal processing unit 112. Signal processing unit 112 calculates a linear correlation coefficient based on the oxygen content signal and the arterial blood pressure signal in a time domain for a plurality of times. In one example, this linear correlation coefficient may be referred to as the cerebral oximeter index (COx). The oxygen content signals transmitted from cerebral oximeter 104 to signal processor 110 can be low pass filtered by anyone of cerebral oximeter 104 itself, signal processing unit 112, or by an intermediate low pass filter in the signal line between cerebral oximeter 104 and signal processing unit 112. Blood pressure monitoring device 108, signal processing unit 112 or an intermediate device in the signal line between blood pressure monitoring device 108 and signal processor 110 can provide low pass filtering of the measured blood pressure signal.

Blood pressure monitoring device 108 may include an intracranial pressure monitoring device (not shown). An intracranial pressure monitoring device may include a catheter-based device which is surgically inserted into patient 102 to directly measure intracranial pressure within the patient's brain. Blood pressure monitoring device 108 may include an arterial blood pressure monitoring device that can be selected from available arterial blood pressure monitoring devices. For example, cerebral oximeter 104 can be a near-infrared spectrometer.

System 100 for diagnosing cerebrovascular autoregulation may also include a display unit 114 that is in communication with signal processing unit 112 to display the linear correlation coefficient values calculated by signal processing unit 112 with respect to other biophysical data of patient 102. For example, display unit 114 may display the linear correlation coefficients calculated as a function of arterial blood pressure. Signal processing unit 112 may determine the cerebral perfusion pressure based on the difference between the arterial blood pressure and the intracranial pressure and provide signals to display unit 114 to display the calculated linear correlation coefficients as a function of the cerebral perfusion pressure.

Cerebral oximeter 104, blood pressure monitoring device 106, display unit 114, and signal processing unit 112 may be communicatively coupled together by any number of wired or wireless communication technologies, including physical wires, fiber optics, or wireless data communications technologies. Signal processing unit 112 can be a stand alone physical component, or may be added as a component to other systems such as to a rack system. Signal processing unit 112 is not necessarily limited to processing only signal data. It may include generally data processing capabilities. In addition, the signal processing operations of signal processing unit 112 may be hard-wired or may be implemented by programming a signal processing unit.

A variety of methods may be employed to determine the state of cerebral autoregulation either statically or dynamically. Each method may include calculating an autoregulation index based on data from multiple measurement sources.

Monitoring Cerebral Autoregulation in a Surgical Environment

In a surgical environment, blood pressure monitoring device 108 will generally be an invasive device for measuring the blood pressure of patient 102. In a surgical environment, blood pressure monitoring device 108 may include an intracranial pressure monitoring device (not shown). An intracranial pressure monitoring device may include a catheter-based device which is surgically inserted into patient 102 to directly measure intracranial pressure within the patient's brain. Blood pressure monitoring device 108 may include an arterial blood pressure monitoring device that can be selected from available arterial blood pressure monitoring devices.

In a surgical environment, it can be beneficial to ensure the accuracy of data acquired from a patient by removing artifacts in real time or near real time. Artifacts, as described below, are generally outlier data or data that is not indicative of the patient.

The cerebral autoregulation index (CAI) is essentially a correlation coefficient between the MAP and the r502 or the NIRS-derived total hemoglobin or blood volume index (BVI). At the present time the CAI measurement during cardiac surgery is performed using the prerecorded ABP and rSO2 NIRS data. This is done off-line because calculation of the correlation coefficient requires removal of artifacts in the real time records of the ABP and the rSO2. The typical artifacts include: transducer flushing, catheter clotting or damping, non-invasive cuff inflation, movement artifacts et cetera. To perform the CAI monitoring all these artifacts should be automatically removed on-line in real time.

There are several methods of artifact removal that can be performed in the real time ABP data. All these methods use the MAP waveform features. The following are examples of atypical MAP waveform features. MAP waveform data meeting one or more of the following criteria may be defined as an artifact and removed.
1) Systolic blood pressure >300 mmHg
2) Diastolic blood pressure <20 mmHg
3) Mean arterial pressure <30 mmHg, or >200 mmHg
4) Pulse Pressure <20 mmHg
5) Heart rate <20 pbm or >200 bpm
6) Slope of ABP wave too large or too small, for example, when the slope of the ABP wave is <−40 mmHg/100 msec

During cardiac surgery, however, there are no ABP waves following initiation of cardiopulmonary bypass (bypass). Thus, these methods are not suitable for the cardiac surgery.

To allow CAI monitoring during cardiac surgery, two different sets of the MAP (and rSO2) features may be used for artifact removal. One set, (for example (1)-(6) for MAP) should be used before bypass and after bypass when the pulse-waves are present. Another set, (for example (2), (6)) should be used during bypass when there are no pulse-waves. Switching of the sets can be done automatically or manually. An automatic switching may employ the lack of the wave's features (1), (2), (4), (5) in the MAP real time data or lack of cardiac electrical activity (R-peaks in the ECG) for a predetermined period of time during bypass. The manual switching may use an Event Marker in the rSO2 cerebral monitor.

The Event Marker is routinely used to manually mark the important events during cardiac surgery. As a signal for the switching between the sets of the ABP artifacts removal features, a marker such as “Clamp on” can be used.

Non-Invasive Methods of Measuring Autoregulation

Described in detail below are numerous systems and methods of non-invasively measuring or creating an index or indices of autoregulation that are an accurate representation of the actual autoregulation. Generally, as discussed below, are non-invasive techniques for measuring or estimating cerebral blood flow and cerebral perfusion pressure. Any combination of these non-invasive techniques can be used to create an autoregulatory index, including any combination of representative indices for components of flow and pressure. For example, non-invasive techniques can be used to measure or estimate both flow and pressure, thereby allowing the creation of an autoregulation index using only non-invasive techniques. As discussed in greater detail below, such complete non-invasive techniques are well-suited for certain applications or patients, such as preterm infants where invasive blood pressure monitoring may be inaccurate or may increase certain risks to the patient.

Exemplary CBF Indices

One suitable method for creating a CBF index is to noninvasively measure red blood cell velocity in the middle cerebral artery using transcranial Doppler (TCD) ultrasound. (See, e.g., Czosnyka M et al., Monitoring of Cerebral Autoregulation in Head-Injured Patients, Stroke 1996; 27(10):1829-34.) Another method, which is invasive, involves using a laser-Doppler probe placed on the brain parenchyma to measure red blood cell flux. (See, e.g., Lam J M et al., Monitoring of Autoregualtion Using Laser Doppler Flowmetry in Patients With Head Injury, J Neurosurg 1997; 86(3):438-45.) Both methods provide signals representative of changes in CBF for determination of an autoregulatory index but both have disadvantages. TCD is technically difficult and cannot be performed in 10-20% of the population due to thick cranial bone. Laser Doppler flowmetry is highly invasive and is usually reserved for only the most severely brain injured patients. A convenient, noninvasive method of measuring changes in CBF for determining an autoregulation index is needed.

Cerebral Oximetry in the Calculation of an Autoregulatory Index

An exemplary autoregulatory index includes a CBF index that uses noninvasive cerebral oximetry to measure cerebral oxygen saturation, as described in patent application WO 2008/097411, incorporated by reference herein. The referenced application describes the correlation of cerebral oxygen saturation measured by near-infrared spectroscopy (NIRS) with spontaneous slow variations in arterial blood pressure (slow waves) to determine an autoregulatory index based on the principle that when cerebral metabolic rate of oxygen is constant, variations in CBF will be reflected in cerebral oxygen saturation (rSO2). For example, sensors 104 and/or 110 may be or include a NIRS sensor.

Cerebral Pulse Contour Analysis in the Calculation of an Autoregulatory Index

A pulsatile signal acquired through the use of NIRS may be used to create a CBF index. This technique is based on the absorption characteristics of hemoglobin. Arterial pulsations caused by the beating heart travel through the circulatory system and can be detected throughout the body. These pulses are dampened significantly when they reach the capillary bed so that venous cardiac pulsations are virtually non-existent. This is the principle behind pulse oximetry which extracts the pulsatile component of NIRS to calculate a wholly arterial oxygen saturation value. Because the vasculature has some compliance, cardiac pulsations cause distention of the arterial bed which increases its blood volume. Since this volume consists entirely of arterial blood, the average oxygen content and oxygen saturation increase during systole. Since NIRS measures optical attenuation due primarily to hemoglobin, arterial distention is reflected as a varying optical signal during each heart beat.

With each beat, the volume of blood rushing into the artery is directly proportional to the actual blood flow rate. The change in hemoglobin volume as a function of time (δI/δt or slope) is directly proportional to blood flow rate. Measurements of the slope, area and shape of the pressure pulse optical signal can be used to derive an index of changes in CBF. Because the process of correlation with arterial pressure slow waves requires only changes over time and not absolute flow rate, this method can accurately provide an autoregulation index based on the CBF change index.

Diffuse Optical Tomography to Indicate Changes in CBF

Diffuse optical tomography is a noninvasive means of measuring changes in CBF. (See, e.g., Culver J P et al., Diffuse Optical Tomography of Cerebral Blood Flow, Oxygenation, and Metabolism in Rat during Focal Ischemia, J Cerebral Blood Flow Metab 2003; 23:911-24.) Collected light is autocorrelated in the time domain to determine red blood cell flux which is used to derive an index of CBF. While not representative of absolute CBF, the CBFi accurately represents changes in CBF. (See, e.g., Durduran T et al., Diffuse Optical Measurement of Blood Flow, Blood Oxygenation, and Metabolism in a Human Brain During Sensorimotor Cortex Activation, Optics Letters 2004; 29(15):1766-8.) This derived CBFi can be filtered and correlated with arterial slow waves to determine an autoregulation index.

This method of measuring red blood cell flux is similar to that used in laser Doppler flowmetry in that diffuse scattering of photons is measured and used to derive a CBFi. The main difference is that diffuse optical tomography is a noninvasive method which is able to measure flow changes through the scalp and skull. It requires the use of multiple detectors placed at several distances from the light source with autocorrelation of the returned signals from the detectors. Beside the advantage of being noninvasive, the sensor used can also be used for cerebral oximetry by adding additional wavelengths of near-infrared light and alternating the measurement of CBFi and rSO2 over time.

Cerebral Perfusion Pressure Indices

There are several noninvasive means to create an index for cerebral perfusion pressure. Variations in blood pressure caused by pulsations of the heart, respiratory waves and slow waves cause changes in the size of peripheral blood vessels. These blood vessel distentions can be measured using arterial tonometry. (See, e.g., Kullo I J, Malik A R, Arterial Ultrasonography and Tonometry as Adjuncts to Cardiovascular Risk Stratification, Journal of the American College of Cardiology 2007; 49(13):1413-26.) With appropriate filtering of the signal, slow variations in arterial distentions measured by arterial tonometry can be correlated with an index of cerebral blood flow to determine autoregulation indices.

In another implementation, the blood vessel distentions may be measured through the use of products employing a servo-controlled blood pressure cuff designed to continuously maintain cuff pressure at the level of the arterial blood pressure. Examples of such products are the Finometer from ADlnstruments, and the Finapres from Finapres Medical Systems. These products provide a noninvasive high fidelity arterial pressure waveform from which variations in arterial pressure pulses caused by slow waves can be derived. The pressure waveform may then be correlated with cerebral oximetry measurement to create an autoregulatory index.

In another implementation, changes in the size of peripheral blood vessels can be measured optically by measuring absorption changes caused by the flux of red blood cells that occur simultaneously with pulsations. In one example, an optical pulse plethysmograph can be used to continuously measure pulsating peripheral blood vessels.

In another implementation, pulse oximeters can measure optical absorption using near-infrared light and display a continuous waveform representing pulsating peripheral blood vessels. Some pulse oximeters, for example, the Masimo Radical product line, can also measure and display variations in pulsation amplitude. The variations are calculated over time and displayed as a pleth variability index (PVI). The pleth waveform used for the calculation of PVI can also be used for correlation with cerebral oximetry measurements to create an autoregulatory index.

An improved implementation includes a second cerebral oximetry sensor 110 placed in a periphery location of the body to acquire a pleth waveform by continuously measuring optical absorption changes in the near-infrared range. Second sensor 110 is placed ideally in an area where pressure changes are maximal such as the palm of the hand or the volar aspect of the forearm. This additional sensor can be used to derive a continuous signal representing peripheral blood vessel distention from which variations in blood pressure caused by slow wave activity can be derived. The variations in vessel distention caused by slow wave activity are extracted from the signal using filtering as previously described and are correlated with cerebral oximetry variations in the time domain, thus deriving a continuously updating correlation coefficient representing the autoregulation state of the patient. This sensor can also be configured to measure peripheral tissue oxygen saturation in addition to vessel distention using the same method as is used for cerebral oximetry. This implementation is an improvement over other methods because both of the measurements are completely noninvasive, both can be performed by a single device, and a measurement of continuous somatic tissue oxygen saturation can be derived.

Heart Rate Variation as an Index for Blood Pressure

There is evidence of the existence in humans of a strong anti-correlation between the oscillations of arterial blood pressure (ABP) and oscillations of heart rate variations (HRV). Such correlation is especially high in the very low frequency band. FIG. 2 is from the article D. Rassi, A Mishin, Time Domain Correlation Analysis of Heart Rate Variability in Preterm Infants, Early Human Development (2005) 81, 341, and illustrates the anti-correlation in preterm infants. FIG. 2 shows simultaneous unfiltered heart rate trace (thick line) and the pulse pressure curve (thin line). Slow oscillations of the HRV coincide inversely with slow oscillations of the pulse pressure curve. The fast oscillations of the pulse pressure curve are caused by respiration.

The exact reason for such strong correlation is not known; however, according to the dominant theory, both the ABP oscillations and HRV oscillations have the same origin—the delay in the baro-reflex feedback loop. Because low frequency heart rate variations are anti-correlated with arterial blood pressure low frequency oscillations, measurements of HRV may be used as the inverse of the blood pressure index.

An example of using a blood pressure index instead of a blood pressure measurement is described in the article A. V. Shemagonov, Testing Dynamic Cerebral Autoregulation without Blood Pressure Monitoring, Journal of Neurological Science 283 (2009). The method described in the article used transcranial Doppler for the cerebral blood flow measurement. In one exmaple, cerebral oximetry measurement of regional oxygen saturation or regional hemoglobin index (blood volume) can be used as an index for CBF instead of transcranial Doppler.

A non-invasive blood pressure index may be based on heart rate variation, as discussed above. Interbeat interval, or the time between consecutive heart beats, may be measured using sensor 110, such as an electrocardiograph (ECG) monitor, or using a pulse oximeter. Heart rate variation may then be determined from the interbeat interval. The low frequency heart rate variation may then be correlated with a CBF index to create an autoregulatory index.

Monitoring Cerebral Autoregulation in Infants Using a Pressure Index

For a preterm infant, invasive blood pressure monitoring by the use of an umbilical cord catheter is the only method to obtain continuous arterial pressure data for use in creating a cerebral autoregulatory index. A non-invasive blood pressure monitor is not suitable because the monitor's sampling rate is too low. For autoregulation monitoring, sampling of ABP should occur at least as often as every five seconds; however, a non-invasive blood pressure monitor samples at best every thirty seconds. There is therefore no reliable method to non-invasively monitor blood pressure in a neonate.

The need for a non-invasive way to monitor preterm infant cerebral autoregulation without using blood pressure monitoring is satisfied in one implementation by using heart rate variation oscillations as an index for blood pressure, as described above. The heart rate variation oscillations may be correlated with the low frequency variations exhibited by a CBF index to create an autoregulatory index. Phase shift between heart rate variation oscillations and low frequency CBF index variations may be used to create an autoregulatory index.

Autoregulation Monitoring with Intermittent Blood Pressure Monitoring

In many cases where an invasive means of blood pressure is considered unsuitable due to the lack of an invasive arterial catheter or when the risks outweigh the benefits of placing a catheter, a proxy for changes in pressure may be used to calculate autoregulation indices (such as changes in heart beat intervals described previously). In these cases a noninvasive sensor 108 and/or 110 for measuring blood pressure may be used, such as by using an occlusive cuff. Most automated cuff pressure devices also have a means to communicate a time-stamped value for blood pressure that can be used to help determine the range of blood pressure where autoregulation is intact by associating past autoregulation indices with previously obtained pressures. These intermittent values can be used to automatically plot correlation coefficients as a function of pressure, enabling the caregiver to determine at a glance whether the blood pressure is too high or too low to support intact autoregulation. Alternatively, if an automated system is not used, the noninvasive autoregulation monitor can alert the staff when autoregulation is impaired, prompting a cuff pressure measurement or invasive pressure monitoring to better understand if the pressure is above or below the accepted normal range.

Noninvasive Cerebral Autoregulation Monitoring as an Early Warning

When the noninvasive autoregulation monitor is in use, it can spontaneously alert the staff to a change in autoregulation that may be related to a change in patient condition that can be traced to some other effect. Examples of these effects include routine assessments or suctioning of the endotracheal tube; certain interventions such as administration of vasoactive drugs, inotropes or surfactant; or feedings. Knowledge of the autoregulation state during these periods can act as a warning to reduce the incidence, modify the dosage, reduce stimulation, add other therapies or more closely follow the patient's condition using increased vigilance or additional monitors.

Noninvasive Cerebral Autoregulation Monitoring to Initiate Invasive Blood Pressure Monitoring

Loss of autoregulation, as assessed by the disclosed example, can indicate a serious deterioration in the patient's condition. As such, it may “tip the scale” in favor of placing an invasive catheter for use with continuous blood pressure monitoring. Once continuous pressure data is available, the caregiver can initiate pressure autoregulation monitoring, using it to more accurately assess the impact of pressure changes on flow.

Mathematical Representations of Cerebral Autoregulation

There are many ways to mathematically represent cerebral autoregulation. The Pearson correlation coefficient is one of the best. The Pearson coefficient refers to the linear relationship between two sets of data. At the point of the lower limit of autoregulation (LLA), the relationship between mean arterial pressure (MAP) and cerebral blood flow (CBF) is highly nonlinear and is dependent on cerebral metabolic rate (CMRO2). Clinically this breakpoint is the most significant point to determine.

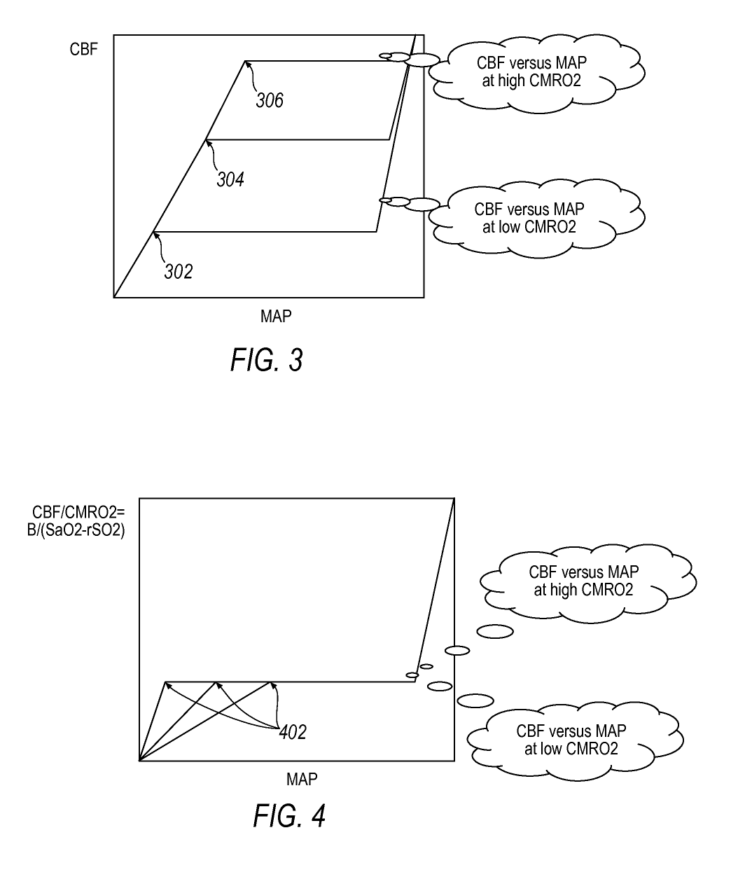

FIG. 3 illustrates CBF versus MAP, which is highly nonlinear at the Lower Limit of Autoregulation (LLA, designated by 302, 304, and 306). CMRO2 is the Cerebral Metabolic Rate of Oxygen Consumption. It represents oxygen demand of the tissue.

To continuously monitor the state of Cerebral Autoregulation (CA) regional cerebral oxygen saturation rSO2 measured by NIRS is commonly used. The rSO2 is defined as the weighted sum of the venous blood oxygen saturation SvO2 and the arterial blood oxygen saturation SaO2:

rSO2=(Vv/Va+Vv)*SvO2+(Va/Vv+Va)*SaO2 (1)

In equation (1), Vv, Va and V=Va+Vv are the venous, arterial and total blood volume in the field of the NIRS sensor, respectively.

The arterial-venous difference SaO2−SvO2, the Cerebral Blood flow (CBF) and the Cerebral Metabolic Rate of Oxygen Consumption (CMRO2) are linked by the Fick equation shown below:

CBF=CMRO2/[SaO2−SvO2]*k*[Hb] (2)

In equation (2), k is the oxygen combining power of hemoglobin (≈1.306 ml of O2 per g of Hb), and [Hb] is the hemoglobin concentration in blood (expressed in g/dL). Using equations (2) and (1) the regional oxygen saturation rSO2 can be expressed as:

rSO2=SaO2−(Vv/V)*[CMRO2/CBF*k*[Hb]] (3)

Equation (3) was fist introduced by I. Tachtsidis in relation to Tissue Oxygenation Index. Because the fraction of venous blood in vessels is ≈0.75 and is relatively constant, equation (3) can be rewritten as:

rSO2=SaO2−A*[CMRO2/CBF] (4)

Equation (4) contains the ratio CBF/CMRO2. Using curves of FIG. 3, autoregulation curves may be depicted in terms of CBF/CMRO2 and MAP at low and high CMRO2 as in FIG. 4. As illustrated in FIG. 4, CBF/CMRO2 versus MAP is highly nonlinear at the point of the LLA, designated by arrows 402.

FIG. 4 illustrates that for CBF/CMRO2 the heights of the auto-regulation plateaus remain constant regardless of tissue oxygen demand and the points of the Low Limit of Autoregulation move only horizontally along these plateaus. Thus, all the auto-regulation curves in FIG. 4 are less scattered on the {CBF/CMRO2; MAP} plane; and using the ratio CBF/CMRO2 for monitoring of autoregulation produces more consistent results. The same is true for rSO2. Because rSO2 and the ratio CBF/CMRO2 are linked to each other by equation (4), using rSO2 for monitoring of autoregulation produces more consistent results than CBF alone.

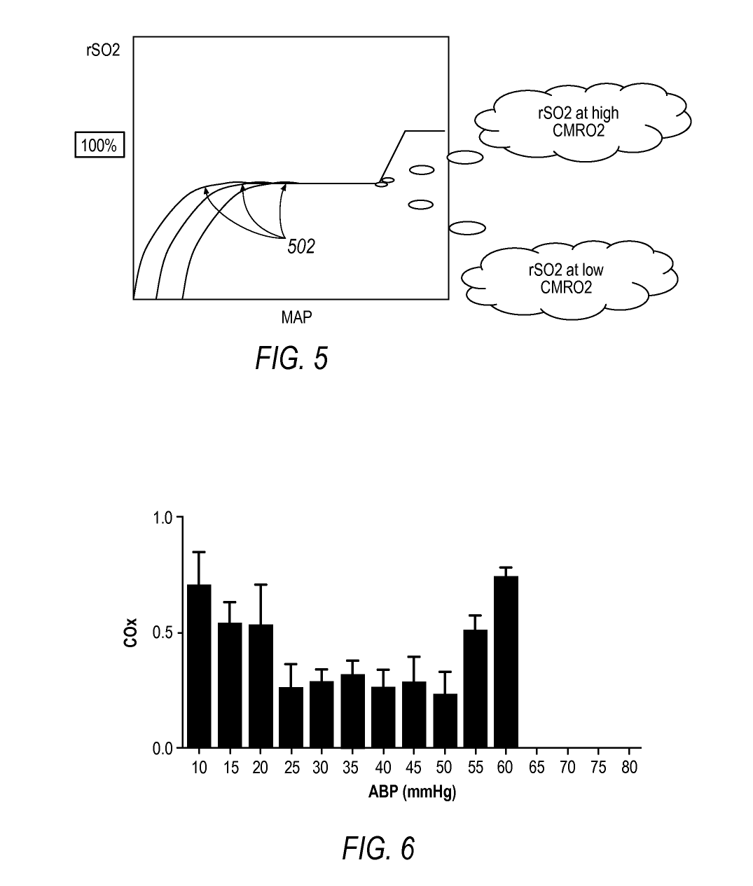

Using equation (4), the autoregulation curves in FIG. 4 can be represented in terms of rSO2 as depicted in FIG. 5. FIG. 5 illustrates that rSO2 versus MAP is highly nonlinear at the point of LLA (designated by arrows 502).

FIG. 5 indicates that if the patient is initially in an autoregulated region, by keeping rSO2 constant the autoregulation state can be maintained regardless of the brain oxygen demand. FIG. 5 further indicates that for neonates, when arterial saturation is highly unstable, the estimation of autoregulation state can use the correlation (SaO2-rSO2; MAP) instead of correlation (rSO2; MAP) that works only when SaO2 is constant.

FIG. 5 illustrates why the limit of autoregulation can be found by analyzing the Pearson correlation coefficient (rSO2; MAP). The Pearson coefficient refers to the linear relationship between the data. Clearly, at the point of the low limit of autoregulation, the relationship between rSO2 and MAP is highly nonlinear. Clinically this point is the most significant point.

Below the point of the low limit of autoregulation the Pearson correlation coefficient (rSO2; MAP) is positive (rSO2 follows MAP). Above the point of the low limit of autoregulation, the Pearson correlation coefficient (rSO2; MAP) is close to zero (rSO2 and MAP oscillate independently).

Referring back, for the situation in which CBF/CMRO2=1/(SaO2−rSO2) the autoregulation chart of FIG. 4 shows a sharper break in the point of the low limit of auto-regulation than does the rSO2 chart of FIG. 5. This means that the correlation coefficient between 1/(SaO2−rSO2) and MAP at the point of the low limit of autoregulation will more sharply fall to zero as pressure decreases.

Reducing Variations Caused by Variations in SaO2

Variations in cerebral oximetry measured oxygen saturation may be caused by changes in arterial oxygen saturation. In some patients arterial oxygen saturation may be below the accepted normal range of 90-100% and/or may vary significantly over time. This is typically true for infants and children with congenital heart defects such as septal defects, persistent patent ductus arteriosus, or other right-to-left shunts where deoxygenated venous blood mixes with oxygenated arterial blood as it is pumped into the systemic circulation. The reduction in arterial oxygen saturation is sometimes referred to as cyanosis as it can impart a bluish tinge to the skin. When arterial saturation is lower than normal, it tends to vary more often and to a greater extent because the arterial saturation range is located on the steeper part of the oxyhemoglobin dissociation curve and small changes in pO2, pH and pCO2 have a greater effect on arterial oxygen saturation. Lower and/or varying arterial saturation levels may also be present in adult patients who have acute respiratory distress syndrome (ARDS), chronic obstructive pulmonary disease (COPD), patients receiving mechanical ventilation or in patients receiving supplemental oxygen therapy. Variations in arterial oxygen saturation can cause parallel changes in cerebral oxygen saturation that can interfere with the measurement of autoregulation.

To minimize this variation in cerebral oxygen saturation (rSO2), continuous monitoring of arterial oxygen saturation (SaO2) using pulse oximetry may be used to calculate a signal representative of arteriovenous oxygen difference, SaO2−rSO2. This difference signal is then processed and correlated with arterial blood pressure in the time domain to produce an index of autoregulation.

Another means of reducing variations caused by fluctuating arterial oxygen saturation may be through the use of fractional tissue oxygen extraction (FTOE) for correlation with arterial blood pressure. FTOE is defined as the arteriovenous oxygen difference (SaO2−rSO2) divided by SaO2 and this calculated parameter can be correlated with arterial blood pressure to produce an index of autoregulation while reducing variations caused by changing SaO2.

Additionally, an estimate of cerebral venous oxygen saturation (SvO2) may be calculated and the correlation of SvO2 with arterial blood pressure measured. Because cerebral tissue is assumed to consist of one quarter arterial blood and three quarters venous blood, cerebral oxygen saturation can be represented as 0.25*SaO2+0.75*SvO2. Therefore, SvO2 can be derived from SaO2 and rSO2 and used to correlate with arterial blood pressure to produce an index of autoregulation while eliminating variations caused by variations in SaO2. SvO2 may be derived from equation (5):

SvO₂=1.33*(rSO₂−0.25*SaO₂) (5)

A System for Measurement

In one example, a NIRS system is described that can continuously measure blood volume pulsations for calculation of CBFi (as described above) using a near-infrared wavelength close to or at the isobestic point for hemoglobin (805 nm) where oxyhemoglobin and unbound hemoglobin absorb equally. This ensures that the measurement of CBFi remains accurate during periods where arterial saturation may be below normal, for example, in patients with cyanosis due to left-right cardiac shunts or other pathology. This system also employs one or more additional wavelengths which are used to measure cerebral oxygen saturation (rSO2). The system is designed to import an invasive blood pressure signal from a primary physiological monitor or can be designed to accept a blood pressure transducer to directly measure blood pressure. The monitor has the capability to display rSO2, systolic, diastolic and mean blood pressure, CBFi and a representation of autoregulation index at multiple blood pressure levels. This display consists of a graph where blood pressure is plotted on the x-axis and the correlation coefficient between blood pressure and CBFi are plotted on the y-axis. This display allows the user to immediately determine the optimal blood pressure range to assure the lowest correlation coefficient and therefore the optimal range to assure autoregulation is intact.

### CONCLUSION

With regard to the processes, systems, methods, heuristics, etc. described herein, it should be understood that, although the steps of such processes, etc, have been described as occurring according to a certain ordered sequence, such processes could be practiced with the described steps performed in an order other than the order described herein. It further should be understood that certain steps could be performed simultaneously, that other steps could be added, or that certain steps described herein could be omitted. In other words, the descriptions of processes herein are provided for the purpose of illustrating certain embodiments, and should in no way be construed so as to limit the claimed invention.

Accordingly, it is to be understood that the above description is intended to be illustrative and not restrictive. Many embodiments and applications other than the examples provided would be apparent upon reading the above description. The scope of the invention should be determined, not with reference to the above description, but should instead be determined with reference to the appended claims, along with the full scope of equivalents to which such claims are entitled. It is anticipated and intended that future developments will occur in the technologies discussed herein, and that the disclosed systems and methods will be incorporated into such future embodiments. In sum, it should be understood that the invention is capable of modification and variation.

All terms used in the claims are intended to be given their broadest reasonable constructions and their ordinary meanings as understood by those knowledgeable in the technologies described herein unless an explicit indication to the contrary in made herein. In particular, use of the singular articles such as “a,” “the,” “said,” etc. should be read to recite one or more of the indicated elements unless a claim recites an explicit limitation to the contrary.

1. A method comprising:
receiving data relating to cerebral blood flow of a patient;
receiving data relating to blood pressure of the patient;
correlating the cerebral blood flow and blood pressure data; and
utilizing the correlated data to monitor a cerebrovascular autoregulation state of the patient.

2. The method of claim 1, further comprising causing a change of blood pressure of the patient based on the cerebrovascular state of the patient determined based on the correlated data.

3. The method of claim 1, wherein the data relating to cerebral blood flow of a patient is at least one of oximeter data, electrocardiogram data, hemoglobin data, and heart rate data.

4. The method of claim 1, wherein the data relating to blood pressure of a patient is at least one of oximeter data, electrocardiogram data, blood pressure data, hemoglobin data, and heart rate data.

5. The method of claim 1, further comprising:
determining a cerebral blood flow measurement based on the received data relating to cerebral blood flow; and
determining a blood pressure measurement based on the received data relating to blood pressure.

6. The method of claim 1, further comprising:
receiving the data relating to cerebral blood flow of the patient utilizing a cerebral oximetry measurement of regional oxygen saturation using a near infrared sensor;
using the measurement as an index to cerebral blood flow; and
receiving the data relating to blood pressure as arterial distension changes as measured by arterial tonometry as an index in changes in blood pressure.

7. The method of claim 1, further comprising:
receiving the data relating to cerebral blood flow of the patient utilizing a cerebral oximetry measurement of regional oxygen saturation using a near infrared sensor;
using the measurement as an index to cerebral blood flow; and
receiving the data relating to blood pressure as signal changes from a non-invasive servo controlled cuff measurement of continuous blood pressure.

8. The method of claim 1, further comprising:
receiving the data relating to cerebral blood flow of the patient utilizing a cerebral oximetry measurement of regional oxygen saturation using a near infrared sensor;
using the measurement as an index to cerebral blood flow; and
receiving the data relating to blood pressure as changes in optical density measured by a non-invasive peripheral optical plethysmograph as an index of blood pressure changes.

9. The method of claim 1, further comprising:
receiving the data relating to cerebral blood flow of the patient utilizing a cerebral oximetry measurement of regional oxygen saturation using a near infrared sensor;
using the measurement as an index to cerebral blood flow; and
receiving the data relating to blood pressure as changes in optical density measured by a non-invasive peripheral pulse oximeter plethysmograph as an index of blood pressure changes.

10. The method of claim 1, further comprising:
receiving the data relating to cerebral blood flow of the patient utilizing a cerebral oximetry measurement of regional oxygen saturation using a near infrared sensor;
using the measurement as an index to cerebral blood flow; and
receiving the data relating to blood pressure as changes in optical density measured by a non-invasive tissue oximeter based on near infrared spectroscopy as in index in changes in blood pressure.

11. The method of claim 1, further comprising:
receiving the data relating to cerebral blood flow of the patient utilizing a cerebral oximetry measurement of regional oxygen saturation using a near infrared sensor;
using the measurement as an index to cerebral blood flow; and
receiving the data relating to blood pressure as changes in heart rate measured by one of electrocardiography and pulse oximetry as an index in changes in blood pressure.

12. The method of claim 1, further comprising:
receiving the data relating to cerebral blood flow of the patient utilizing a cerebral oximetry measurement of changes in optical density resulting from cardiac pulsations using a near infrared sensor;
using the measurement as an index to cerebral blood flow; and
receiving the data relating to blood pressure from an invasive measurement of blood pressure of the patient.

13. The method of claim 1, further comprising:
receiving the data relating to cerebral blood flow of the patient utilizing a cerebral oximetry measurement of changes in optical density resulting from cardiac pulsations using a near infrared sensor;
using the measurement as an index to cerebral blood flow; and
receiving the data relating to blood pressure as arterial distension changes as measured by arterial tonometry as an index in changes in blood pressure.

14. The method of claim 1, further comprising:
receiving the data relating to cerebral blood flow of the patient utilizing a cerebral oximetry measurement of changes in optical density resulting from cardiac pulsations using a near infrared sensor;
using the measurement as an index to cerebral blood flow; and
receiving the data relating to blood pressure as signal changes from a non-invasive servo controlled cuff measurement of continuous blood pressure.

15. The method of claim 1, further comprising:
receiving the data relating to cerebral blood flow of the patient utilizing a cerebral oximetry measurement of changes in optical density resulting from cardiac pulsations using a near infrared sensor;
using the measurement as an index to cerebral blood flow; and
receiving the data relating to blood pressure as changes in optical density measured by a non-invasive peripheral optical plethysmograph as an index of blood pressure changes.

16. The method of claim 1, further comprising:
receiving the data relating to cerebral blood flow of the patient utilizing a cerebral oximetry measurement of changes in optical density resulting from cardiac pulsations using a near infrared sensor;
using the measurement as an index to cerebral blood flow; and
receiving the data relating to blood pressure as changes in optical density measured by a non-invasive peripheral pulse oximeter plethysmograph as an index of blood pressure changes.

17. The method of claim 1, further comprising:
receiving the data relating to cerebral blood flow of the patient utilizing a cerebral oximetry measurement of changes in optical density resulting from cardiac pulsations using a near infrared sensor;
using the measurement as an index to cerebral blood flow; and
receiving the data relating to blood pressure as changes in optical density measured by a non-invasive tissue oximeter based on near infrared spectroscopy as in index in changes in blood pressure.

18. The method of claim 1, further comprising:
receiving the data relating to cerebral blood flow of the patient utilizing a cerebral oximetry measurement of changes in optical density resulting from cardiac pulsations using a near infrared sensor;
using the measurement as an index to cerebral blood flow; and
receiving the data relating to blood pressure as changes in heart rate measured by one of electrocardiography and pulse oximetry as an index in changes in blood pressure.

19. The method of claim 1, further comprising:
receiving the data relating to cerebral blood flow of the patient utilizing a near infrared measurement of red blood cell movement using diffused optical tomography;
using the measurement as an index to cerebral blood flow; and
receiving the data relating to blood pressure from an invasive measurement of blood pressure of the patient.

20. The method of claim 1, further comprising:
receiving the data relating to cerebral blood flow of the patient utilizing a near infrared measurement of red blood cell movement using diffused optical tomography;
using the measurement as an index to cerebral blood flow; and
receiving the data relating to blood pressure as arterial distension changes as measured by arterial tonometry as an index in changes in blood pressure.

21. The method of claim 1, further comprising:
receiving the data relating to cerebral blood flow of the patient utilizing a near infrared measurement of red blood cell movement using diffused optical tomography;
using the measurement as an index to cerebral blood flow; and
receiving the data relating to blood pressure as signal changes from a non-invasive servo controlled cuff measurement of continuous blood pressure.

22. The method of claim 1, further comprising:
receiving the data relating to cerebral blood flow of the patient utilizing a near infrared measurement of red blood cell movement using diffused optical tomography;
using the measurement as an index to cerebral blood flow; and
receiving the data relating to blood pressure as changes in optical density measured by a non-invasive peripheral optical plethysmograph as an index of blood pressure changes.

23. The method of claim 1, further comprising:
receiving the data relating to cerebral blood flow of the patient utilizing a near infrared measurement of red blood cell movement using diffused optical tomography;
using the measurement as an index to cerebral blood flow; and
receiving the data relating to blood pressure as changes in optical density measured by a non-invasive peripheral pulse oximeter plethysmograph as an index of blood pressure changes.

24. The method of claim 1, further comprising:
receiving the data relating to cerebral blood flow of the patient utilizing a near infrared measurement of red blood cell movement using diffused optical tomography;
using the measurement as an index to cerebral blood flow; and
receiving the data relating to blood pressure as changes in optical density measured by a non-invasive tissue oximeter based on near infrared spectroscopy as in index in changes in blood pressure.

25. The method of claim 1, further comprising:
receiving the data relating to cerebral blood flow of the patient utilizing a near infrared measurement of red blood cell movement using diffused optical tomography;
using the measurement as an index to cerebral blood flow; and
receiving the data relating to blood pressure as changes in heart rate measured by one of electrocardiography and pulse oximetry as an index in changes in blood pressure.

26. The method of claim 1, further comprising:
receiving the data relating to cerebral blood flow of the patient utilizing pulse contour analysis on a signal received from a near infrared sensor to measure characteristics of the pulsatile component of total hemoglobin;
using the measurement as an index of cerebral blood flow resulting from arterial oscillations; and
calculating an index corresponding to cerebral blood flow.

27. The method of claim 1, further comprising:
receiving the data relating to cerebral blood flow of the patient utilizing a cerebral oximetry measurement of regional oxygen saturation using a near infrared sensor;
monitoring arterial oxygen saturation of the patient; and
normalizing the regional oxygen saturation data based on the monitored arterial oxygen saturation of the patent.

28. The method of claim 1, further comprising:
receiving the data relating to cerebral blood flow of the patient utilizing a cerebral oximetry measurement of regional oxygen saturation using a near infrared sensor; and
calculating fractional tissue oxygen extraction as an index of cerebral blood flow.

29. The method of claim 1, further comprising:
receiving the data relating to cerebral blood flow of the patient utilizing a cerebral oximetry measurement of regional oxygen saturation using a near infrared sensor; and
calculating venous oxygen saturation based on the measurement of regional oxygen saturation and arterial oxygen saturation as an index of cerebral blood flow.

30. The method of claim 1, further comprising:
receiving the data relating to cerebral blood flow of the patient utilizing a near infrared measurement of total hemoglobin as an index of cerebral blood flow.

31. A method comprising:
receiving data relating to cerebral blood flow of a patient;
receiving data relating to blood pressure of the patient;
identifying a methodology of data collection used to collect the data relating to cerebral blood flow and blood pressure;
setting limits of outlier data based the identified methodology of data collection;
identifying outlier data based on the limits;
excluding the outlier data;
correlating the non-excluded cerebral blood flow and blood pressure data; and
utilizing the correlated data to monitor a cerebrovascular autoregulation state of the patient.

32. The method of claim 31, further comprising adjusting the limits of outlier data based on at least one patient characteristic.

33. The method of claim 32, wherein the at least one patient characteristic is a location of the patient within a hospital, age, body size, a level of acuity, a level of disease, whether the patient is going on bypass, and whether the patient will be experiencing hypothermia.
